(19) United States
(12) Patent Application Publication
(10) Pub. No.: US 2010/0104522 A1
(43) Pub. Date: Apr. 29, 2010

(54) COMPOSTION FOR SKIN EXTERNAL USE CONTAINING OMEGA-3 FATTY ACID

(75) Inventors: JunCheol CHO, Soowon-city (KR); ChanKoo Kang, Seoul (KR); SangHoon Han, Suwon city (KR); Eui-Dong Son, Yongin-city (KR); HakHee Kang, Seongnam-city (KR)

Correspondence Address: MERCHANT & GOULD PC, P.O. BOX 2903, MINNEAPOLIS, MN, 55402-0903, US

(73) Assignee: Amorepacific Corporation, Seoul (KR)

(21) Appl. No.: 12/683,916

(22) Filed: Jan. 7, 2010

Related U.S. Application Data
(62) Division of application No. 11/928,669, filed on Oct. 30, 2007.
Application No. 11/928,669 is a continuation-in-part of application No. 11/840,512, filed on Aug. 17, 2007, now abandoned.

(30) Foreign Application Priority Data
Sep. 1, 2006 (KR) 10-2006-0084425

(51) Int. Cl. A61K 8/36; A61K 31/202; A61K 36/00; A61K 38/02; A61P 17/00; A61Q 17/04

(52) U.S. Cl. 424/59; 514/560; 424/725; 514/6

(57) ABSTRACT

Stable preparations containing omega-3 fatty acid as an effective ingredient are disclosed. Further, uses of omega-3 fatty acid in preventing or alleviating skin aging, moisturizing skin, improving skin roughness, restoring or strengthening skin barrier function, improving skin elasticity, recovering or preventing skin damage induced by UV are disclosed.

### CROSS-REFERENCE TO RELATED APPLICATIONS

This application is a Divisional application of U.S. Ser. No. 11/928,669 filed on Oct. 30, 2007, which is a Continuation-In-Part of United Ser. No. 11/840,512 filed on Aug. 17, 2007, which claims the benefit of Korean Patent Application No. 10-2006-0084425 filed on Sep. 1, 2006, the disclosures of which applications are incorporated herein by reference. A claim of priority to all, to the extent appropriate is made.

### BACKGROUND OF THE INVENTION

1. Field of the Invention

The present invention relates to a composition for skin external use containing omega (ω)-3 fatty acid as an effective ingredient.

2. Description of the Prior Art

The skin has a barrier function against physicochemical stimulus and microbe invasion between extracorporeal external environment and in vivo organs. The keratin layer, which is the outermost layer of the skin, performs the barrier function of the skin and the lipid bilayer of the skin performs an auxiliary function. For the purpose of maintaining elasticity and flexibility of the skin, it is very important for the keratin layer to maintain an appropriate amount of water. The normal skin contains about 30% of water or water-soluble ingredients. When the water content of the keratin layer is lowered to 10% or less, dry skin symptoms are expressed and the skin is cracked or becomes rough. The causes of weakening the barrier function of the skin are divided into internal and external causes. The internal causes may include diseases, genetic defects, aging and the like. The external causes may include the use of soap or cleansing agent, UV light, long-term exposure in temperature and humidity environments of unfavorable conditions, unhealthy eating habits and the like. Due to the internal and external causes, the functions of the keratin layer serving as a barrier are structurally and physiologically degenerated, so that the barrier function is weakened. Therefore, to maintain the appropriate water is basic and essential for the physiology of the skin so that the water of the keratin layer is not lowered below an optimum level.

In addition, the aging of the skin can be easily observed, so that interests are increased. The representative symptoms, which are expressed as the skin ages, include the increase of the skin wrinkles, the decrease of the skin elasticity, the decrease of the skin gloss, the roughening of skin, the yellows of the skin, the partial pigmentation and the like. The skin aging can be divided into natural aging attributable to the internal causes and photo aging attributable to the external causes, particularly sunlight. The photo aging that is expressed on face, the nape of the neck and the back of the hand, which are much exposed to the sunlight, thickens the skin and accumulates the denaturalized elastic fibers. To the contrary, in the parts that are not exposed to the sunlight, the skin is shrunken due to the natural aging progress, the function is deteriorated and the skin thickness is decreased.

In the early of 1970's, it was known that the Eskimos residing at the Greenland hardly had the blood vessel related diseases such as heart disease, cancer, diabetes and the like. Therefore, the researches on the eating menu have been performed. It was found that the Eskimos ate, two times or more every day, the meats in the deep sea such as fur seal, small whale and the like. The researches on the omega-3 (ω-3) fatty acid contained in the meats have been performed again and again. In the 1980's, the researches on the general effects of the polyunsaturated fatty acid (PUFA), especially ω-3 fatty acid have been intensively performed. To date, it has been known that the unsaturated fatty acids are essential for the normal growth and development and play an important role in the prevention and treatment of the coronary artery disease, hypertension, arthritis, autoimmune disorders, cancers and the like.

The ω-3 fatty acid that is known as an ingredient good to the body has never been used in the cosmetics. The reason is as follows: the excellent effect of the ω-3 fatty acid on the skin has not been perceived and the ω-3 fatty acid has a serious drawback that it is easily degenerated at room temperatures or in the formulations. Therefore, it is seriously needed a means capable of stably maintaining the ω-3 fatty acid at room temperatures or in the formulations and effectively applying the ω-3 fatty acid to the skin.

### SUMMARY OF THE INVENTION

Accordingly, an embodiment of the present invention has been made to solve the above problems. An object of an embodiment of the invention is to restore and strengthen the damaged and disordered skin barrier and to put life into the tired and depleted cell.

Another object of an embodiment of the invention is to protect the skin from external harmful environments and to prevent the skin water from being emitted to an outside.

Still another object of an embodiment of the invention is to defend the skin against the harmful factors in the skin and to regenerate collagen in the damaged and depleted skin.

Yet still another object of an embodiment of the invention is to increase the absorption of effective ingredients into the skin and to correctly transport the effective ingredients to a target part in the skin.

Another object of an embodiment the invention is to provide a stable formulation having omega-3 fatty acid as an effective ingredient.

In order to achieve the above object, there is provided a composition for skin external use containing ω-3 fatty acid as an effective ingredient.

Hereinafter, the invention will be more specifically described.

A composition for skin external use according to an embodiment of the invention is preferably an oil in water emulsion formulation. Herein, the ω-3 fatty acid may be dissolved or mixed on an oil phase of the oil in water emulsion. In addition, the ω-3 fatty acid may exist in lipid nano-particles that are added in a water phase of the oil in water emulsion.

A composition for skin external use according to an embodiment of the invention is an oil in water emulsion, and the oil in water emulsion may consist of oil droplets containing the ω-3 fatty acid and a water phase including lipid nano-particles that do not contain the ω-3 fatty acid.

A composition for skin external use according to an embodiment of the invention is an oil in water emulsion, and the oil in water emulsion may consist of oil droplets containing the ω-3 fatty acid and a water phase including lipid nano-particles containing the ω-3 fatty acid.

In the mean time, in the composition for skin external use according to an embodiment of the invention, the ω-3 fatty acid may be contained in the lipid nano-particles and the lipid nano-particles may be dispersed in water.

In the mean time, in the composition for skin external use according to an embodiment of the invention, the ω-3 fatty acid may be contained in liposome that is dispersed in water or aqueous medium.

In the specification including the claims, the “ω-3 fatty acid” is a kind of unsaturated fatty acids, in which a methyl group, which is an end of a COOH group in a chemical structure of the fatty acid, is denoted as a Greek alphabet “ω”, and it is named in accordance with the position of the first double bond from the methyl group. An example of the ω-3 fatty acid includes EPA (eicosapentanoic acid, C20:5ω-3), DPA (docosapentaenoic acid), DHA (docosahexanoic acid, C22:6ω-3), ALA (α-Linolenic acid, C18:3ω-3) and the like.

A preferred formulation according to an embodiment of the invention is oil in water (o/w) emulsion and emulsion including lipid nano-particles. Herein, the lipid nano-particle is a particle having a nm size and means a particle consisting of lipid or a lipid mixture, in which an inside matrix of the particle is solid at room temperatures and in the human body. The ω-3 fatty acid that is an effective ingredient of the composition for the skin external use according to the invention may be dissolved in the oil of the emulsion or inserted in the particle matrix of the lipid nano-particle. In addition, the ω-3 fatty acid may be dissolved in the oil phase of the emulsion and inserted in the matrix of the lipid nano-particle at the same time.

According to an embodiment of the invention, the ω-3 fatty acid may be contained in the form of black currant oil (BCO). The black currant oil contains a high content of the ω-3 fatty acid. In addition, the ω-3 fatty acid from a source originated from an animal, for example fishes, and synthesized or semi-synthesized ω-3 fatty acid may be used.

According to an embodiment of the invention, a preferred type of the ω-3 fatty acid is black currant oil. In other words, instead of the ω-3 fatty acid, the black currant oil may be loaded to the lipid nano-particles or mixed in the oil phase of the oil in water emulsion. The place of origin of the black currant is northwest Europe and the black currant grows well in the shade and is a hardy place. A height thereof reaches about 1 m. A flower thereof is a hermaphrodite flower, comes into full bloom in April˜May and constitutes raceme. A fruit thereof is a juicy fleshy fruit and ripens in July˜August. A color of the fruit is red, so that it is also referred to as red currant. The black currant is R. nigrum whose origin place is Europe and Central Asia. The black currant has much juice and is very sour, so that it is used as jam, juice or jelly in Europe. In addition, the black currant is often fermented and is made as a drug tablet. The currant much contains vitamin C, especially, and calcium, phosphorous, iron and the like. The term “currant” may also indicate “small dry grape having no seed.” The black currant much contains potassium, magnesium, iron, calcium, vitamin A, vitamin B, vitamin C, a very small amount of minerals, organic acid, poyphen more than any other fruits, so that it is referred to as “king of berries.” It much contains natural activated plant nutrient complex (phytonutrients) that plays an important role in the health conservative. In particular, it contains the most content of anthocyanin, as compared to the other fruits, and the oil thereof plentifully contains the ω-3 and ω-5 essential fatty acid.

According to an embodiment of the invention, the oil in water (o/w) emulsion to which the ω-3 fatty acid is loaded may be simply prepared by mixing the black currant oil with the oil phase of the emulsion and then emulsifying it in the water. An example of an equipment for dispersing the oil phase may include a high speed stirrer, a static blender, an ultrasonic or high pressure homogenizer and the like.

In the composition for skin external use according to an embodiment of the invention, a content of the ω-3 fatty acid is preferably 0.01˜50 wt %, more preferably 0.01˜10 wt %, most preferably 0.01˜5 wt %. The most preferred content is 0.5˜3 wt %. When the content is less than the above content, efficacy is slight. Even when the content is more than the above content, the efficacy is not much increased and is thus uneconomical and a formulation may be unstable.

The lipid nano-particle to which the ω-3 fatty acid is loaded according to an embodiment of the invention may be prepared by melting lipid at about 5° C., which is above the melting point thereof, heating the BCO to the same temperature and then mixing the BCO with the melted lipid. The lipid melt containing the ω-3 fatty acid is dispersed in a high-temperature surfactant solution of the same temperature. For the dispersion of the lipid melt, any means may be used as long as the means can provide energy enough to disperse the melted phase into a particle size less than 50 μm, preferably 10 μm, more preferably 5 μm, most preferably 1 μm (i.e., lipid nano-particle). The same equipment as described in the emulsion preparation may be also used. In particular, the ultrasonic and high pressure homogenizer (for example, piston-gap homogenizer, jet stream homogenizer) is most preferable so as to prepare the lipid nano-particles. As a result of the dispersion process, nano-emulsion of high temperature is obtained. When the nano-emulsion is cooled, the lipid is re-crystallized, so that solid lipid micro-particles or lipid nano-particles are obtained.

The lipid, which is used so as to mix the ω-3 fatty acid or to mix the oil containing the ω-3 fatty acid, may be a single solid lipid, a mixture of two or more solid lipids or a mixture of one or more liquid lipids and solid lipids. The lipid may be plant wax such as carnauba and candelilla, fatty acid derivatives such as cetyl palmitate, bio-lipid found in organisms such as ceramide, cholesterol and phosphatidylcholine. An example of the surfactant may include Tego Care 450 (Goldschmidt GmbH, Germany) and Planta Care 2000 (Cognis GmbH, Germany).

In order to prepare a final local formulation, the lipid particle suspension is mixed to oil in water (o/w) cream (or lotion, i.e., formulation having reduced the oil content) at room temperatures. Preferably, the lipid particle suspension has a relatively high concentration (for example, 30%, 50%) so that the oil in water cream or lotion is not too strongly diluted.

In the composition for skin external use according to an embodiment of the invention, the ω-3 fatty acid becomes a constitutional ingredient of a cell membrane and intercellular lipid and takes charge of flexibility and material transfer between the inside and outside of the cell, and acts like a flexible spring of a bed. In other words, a proper unsaturated fatty acid provides the skin with flexibility and elasticity. When it is deficient in the skin, the skin becomes hard and rough and is apt to be damaged. In the skin, the ω-3 fatty acid may perform a variety of functions such as skin barrier, moisturization and flexibility.

In the composition for skin external use according to an embodiment of the invention, the lipid nano-particles exhibit an occlusive effect, thereby increasing permeation of active ingredients. In order to achieve the effect, the lipid nano-particles to which the ω-3 fatty acid is not loaded may be mixed into oil in water cream or lotion in which the ω-3 fatty acid is contained in the emulsion oil droplets. The occlusive effect of the lipid nano-particles promotes the adsorption of the ω-3 fatty acid into the skin from the oil in water emulsion system.

According to an embodiment of the invention, the ω-3 fatty acid may be transported to the skin through the other skin transport system, for example liposome, multiple emulsion, micro-sponge transport system or cochleate.

The composition for skin external use according to an embodiment of the invention may further comprise Rosevita extract and/or copper-peptide as an effective ingredient.

The Rosevita extract is also referred to as Rosa Davurica bud extract. The

Rosa Davurica bud is a kind of wild roses, is a floral tribute plant in the end of Cretaceous period and has a strong life force, in view of the fossil that was formed before 3,500 million years ago. The fruit and leaf of Rosa Davurica bud contain vitamin C 20˜60 times higher than the lemon and contain a large amount of beta carotin. In particular, it contains a high concentration of catechin, so that it has a strong effect of recovering the skin that is damaged due to the oxidative stress of various harmful elements of the interior and exterior of the skin, such as ultraviolet.

The copper-peptide is a low molecular peptide containing Cu, preferably having a molecular weight of 500 or less, or having 5 and less amino acids. For example, copper tripeptide-1 (GHK sequence) (molecular weight: approximately 420) may be used. The copper-peptide promotes the collagen production of the internal skin cells and treats the damage of the skin tissue.

In the composition for skin external use according to an embodiment of the invention, the ω-3 fatty acid, which is an effective ingredient, has a function of recovering and strengthening the damaged and disordered skin barrier and a nutrition function of putting life into the tired and depleted cell. The function of the ω-3 fatty acid is a healing function. In addition, the lipid nano-particle remains attached to the skin even after the ω-3 fatty acid is emitted, so that it protects the skin from the external harmful environment and prevents the skin water from being emitted to the outside. The function of the lipid nano-particle is a sealing function. The Rosevita extract, which is an additional effective ingredient, defends the skin against the harmful factors in the skin and the copper-peptide regenerates the collagen in the damaged and depleted skin. Like this, in an embodiment of the invention, the four important ingredients perform the inherent functions thereof, thereby exhibiting a synergetic effect of protecting the skin as a whole.

The lipid nano-particle according to an embodiment of the invention increases the absorption of the effective ingredients into the skin and enables the effective ingredients to be correctly transported to a target part in the skin.

FIG. 1 shows a particle according to an embodiment of the invention, i.e., complex (nano lipid carrier) in which ω-3 fatty acid is dispersed in a lipid particle. The ω-3 fatty acid may be mixed with the Rosevita extract and/or the copper peptide and dispersed in the lipid particle.

### BRIEF DESCRIPTION OF THE DRAWINGS

The above and other objects, features and advantages of the present invention will be more apparent from the following detailed description taken in conjunction with the accompanying drawings, in which:

FIG. 1 shows a particle according to an embodiment of the invention, i.e., complex (nano lipid carrier) in which ω-3 fatty acid is dispersed in a lipid particle;

FIG. 2 is an electron microscope photograph of lipid particles prepared according to an embodiment of the invention;

FIG. 3 shows an experiment result showing an effect of ω-3 fatty acid on MMP-1 generation inhibition;

FIG. 4 shows a measured result of skin water contents before and after a composition for skin external use according to an embodiment of the invention is applied;

FIG. 5 shows a measured result of keratin amounts before and after a composition for skin external use according to an embodiment of the invention is applied;

FIG. 6 shows skin roughness before and after a composition for skin external use according to an embodiment of the invention is applied;

FIG. 7 shows compact degrees of the dermis before and after a composition for skin external use according to an embodiment of the invention is applied;

FIG. 8 shows a comparison result of skin permeation of a transport system according to an embodiment of the invention and another system;

FIG. 9 shows a comparison result of effect continuation of a transport system according to an embodiment of the invention and another system; and

FIG. 10 shows a comparison result of water contents of a transport system according to an embodiment of the invention and another system.

FIG. 11 is a graph showing the effect of omega-3 fatty acid on ceramide generation.

FIG. 12 is a graph showing the effect of omega-3 fatty acid on elastin generation.

FIG. 13 is a graph showing the effect of omega-3 fatty acid on MMP amounts.

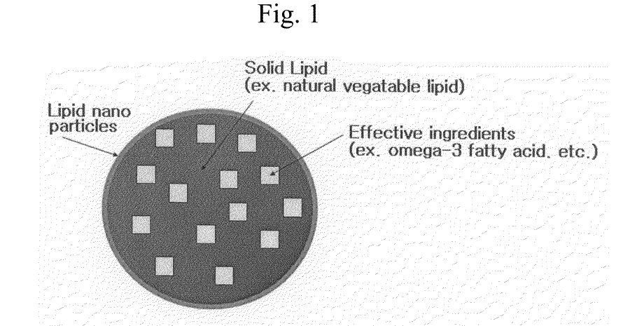

### DETAILED DESCRIPTION OF THE PREFERRED EMBODIMENTS

Hereinafter, the invention will be more specifically described with reference to the embodiments. However, it should be noted that the invention is not limited thereto.

### EXAMPLES

### Example 1

### Preparation of Lipid Nano-Particles Containing the ω-3 Fatty Acid

Carnauba wax was melted at about 5° C. or higher from the melting point, which is above the melting point thereof, and the black currant oil was heated to the same temperature, which was then mixed with the melted lipid phase. The lipid melt containing the ω-3 fatty acid was dispersed in the high-temperature surfactant (Tego Care 450) (Goldschmidt GmbH, Germany) solution of the same temperature. In order to form a particle size less than 1 μm, it was dispersed using a jet stream homogenizer. The resulting nano emulsion of high temperature was cooled to re-crystallize the lipid, thereby preparing the solid lipid nano-particles.

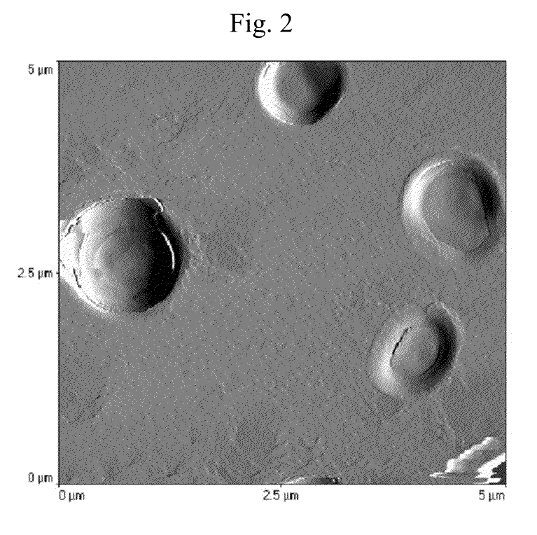

A photograph of the prepared nano-particles is shown in FIG. 2.

### Example 2

### Preparation of Lipid Nano-Particles without the ω-3 Fatty Acid

Lipid nano-particles were prepared in the same manner as the above Example 1 except that ω-3 fatty acid was not added.

### Example 3

### Preparation of Oil in Water Emulsion Consisting of Oil Droplets Containing ω-3 Fatty Acid and a Water Phase Including Lipid Nano-Particles which do not Contain ω-3 Fatty Acid

An emulsion formulation was prepared according to the composition of Table 1 below. The oil phase components were uniformly dissolved and mixed at 70□ or higher. The aqueous phase components 1 were uniformly dissolved and mixed at 70□ or higher except the lipid nano-particles. The oil phase components were slowly added to the batch of the aqueous phase components 1 while mixing them using a homogenizer. After mixing them for 3 minutes, the aqueous phase component 2 was slowly added to the mixture, and they were mixed using a homogenizer for the second emulsification and slowly cooled. When the temperature of the emulsion became 50° C. or lower, the lipid nano-particles prepared according to the above Example 2 were slowly added to the mixture while paddle-mixing.

### Example 4

### Preparation of Oil in Water Emulsion Consisting of Oil Droplets Containing ω-3 Fatty Acid and a Water Phase Including Lipid Nano-Particles Containing ω-3 Fatty Acid

An emulsion formulation was prepared according to the composition of Table 1 below. The emulsion was prepared in the same manner as the above Example 3 except that lipid nano-particles prepared by the above Example 1 were added.

### Example 5

### Preparation of an Emulsion in which Lipid Nano-Particles Containing ω-3 Fatty Acid are Dispersed in Water

An emulsion formulation was prepared according to the composition of Table 1 below. The emulsion was prepared in the same manner as the above Example 4 except that black currant oil (ω-3 fatty acid) is not contained in the oil phase components.

### Example 6

### Preparation of an Emulsion in which ω-3 Fatty Acid is Contained in a Liposome Dispersed in Water or an Aqueous Medium

An emulsion formulation was prepared according to the composition of Table 1 below. Liposome was prepared according to a method well known in the art as follows: ω-3 fatty acids were dissolved in ethanol. Phospholipids was dissolved in the aqueous phase at 70° C. or higher. The ethanol part were put into the aqueous phase while uniformly mixing them and slowly cooled to form liposome. The emulsion was prepared in the same manner as the above Example 5 except that a liposome base containing ω-3 fatty acids was used instead of lipid nano-particle dispersion.

TABLE 1

| Phase | Components | Example 3 | Example 4 | Example 5 | Example 6 |
|---|---|---|---|---|---|
| Oil phase | Stearic acid | 1.0 | 1.0 | 1.0 | 1.0 |
| | Cetearyl alcohol | 0.2 | 0.2 | 0.2 | 0.2 |
| | Hydrogenated oil | 0.7 | 0.7 | 0.7 | 0.7 |
| | Hydrophobic glyceryl stearate | 0.5 | 0.5 | 0.5 | 0.5 |
| | Hydrogenated lecithin/C12-16 alcohol/ | 1.0 | 1.0 | 1.0 | 1.0 |
| | palmitic acid | | | | |
| | Preseratives | 0.3 | 0.3 | 0.3 | 0.3 |
| | Polydecen | 4.0 | 4.0 | 4.0 | 4.0 |
| | meadow form seed oil | 1.0 | 1.0 | 1.0 | 1.0 |
| | Pentaerythrityl tetraisostearate | 3.0 | 3.0 | 3.0 | 3.0 |
| | Dimethicone | 0.5 | 0.5 | 0.5 | 0.5 |
| | Cetyl octanoate | 2.0 | 2.0 | 2.0 | 2.0 |
| | Black currant oil(BCO, ω-3) | 1.0 | 1.0 | — | — |
| | Cyclomethicone | 3.0 | 3.0 | 3.0 | 3.0 |
| | Fragrance | Proper | Proper | Proper | Proper |
| | | amound | amound | amound | amound |
| Aqueous | Purified water | To 100 | To 100 | To 100 | To 100 |
| phase 1 | Glycerin | 4.0 | 4.0 | 4.0 | 4.0 |
| | Butylene glycol | 5.0 | 5.0 | 5.0 | 5.0 |
| | Disodium EDTA | 0.02 | 0.02 | 0.02 | 0.02 |
| | TEA (Triethanolamine) | 0.15 | 0.15 | 0.15 | 0.15 |
| | Preservatives | 0.2 | 0.2 | 0.2 | 0.2 |
| | Dispersion of lipid nano-particles without ω-3 | 10.0 | — | — | — |
| | fatty acid | | | | |
| | Dispersion of lipid nano-particles containing ω-3 | — | 10.0 | 10.0 | — |
| | fatty acid | | | | |
| | Liposome containing ω-3 fatty acid | — | — | — | 10.0 |
| Aqueous | Carbomer 1% aqueous solution | 12.0 | 12.0 | 12.0 | 12.0 |
| phase 2 | | | | | |

### Example 6

### A Rosevita Extract and Cooper Peptide

A formulation containing Rosevita extract and cooper peptide in addition to omega-3 fatty acid as effective ingredients was prepared according to the Table 2 below. The dispersion of lipid nano-particles containing ω-3 fatty acid in Table 2 is the lipid nano-particles prepared according to the above Example 1. After uniformly mixing the oil phase components with the aqueous phase components at 70° C. or higher using a homogenizer, the mixture was cooled. When the temperature of the mixture became 50° C. or lower, the dispersion of lipid nano-particles containing ω-3 fatty acid, the Rosevita extract and cooper peptide were put and slowly paddle-mixed to form an emulsion formulation.

TABLE 2

| Components | Example 6 |
|---|---|
| Purified water | To 100 |
| Dispersion of lipid nano-particles | 10.0 |
| containing ω-3 fatty acid | |
| Rosevita | 3.0 |
| Cooper peptide | 3.0 |
| EDTA 2Na | 0.02 |
| Glycerin | 3.00 |
| Plant hydrogenated oil | 1.50 |
| Stearic acid | 0.60 |
| Glyceryl stearate | 1.00 |
| Cetearyl alcohol | 2.00 |
| arachidyl behenyl alcohol & arachidyl glucoside | 1.00 |
| Cetearyl alcohol & cetearyl glucoside | 2.00 |
| Liquid paraffin | 6.00 |
| Caprylic/capric triglyceride | 6.00 |
| Carbomer | 0.05 |
| Triethanolamine | 0.05 |
| Preservatives, fragrances, colorant | Proper amount |

Hereinafter, the inventors carried out the experimentations below in order to demonstrate the effect of the present invention.

### Experimental Example 1

### Test of an Effect of ω-3 Fatty Acid on MMP-1 Production Inhibition

Normal human fibroblasts were divided and put into a 24-well plate in an amount of 1×10⁵ cells per well. The cells were grown in a medium containing 10% serum for 24 hours and then were further grown in a medium without 10% serum for 24 hours. After washing them with PBS once, 100 μL PBS was added and 30 mJ/cm² UVB was radiated to them. After removing PBS, they were treated with omega-3 fatty acid in concentrations of 0, 1, 5 and 10 mM by inserting 1 ml per well in a new medium without serum. After 48 hours from the treatment of omega-3 fatty acids, supernatant liquor was recovered and a part thereof was subject to MMP-1 expression analysis using an ELISA kit (amersharm pharmacia, cat. #; RPN 2610). The amounts of MMP-1 measured were modified to total protein amounts.

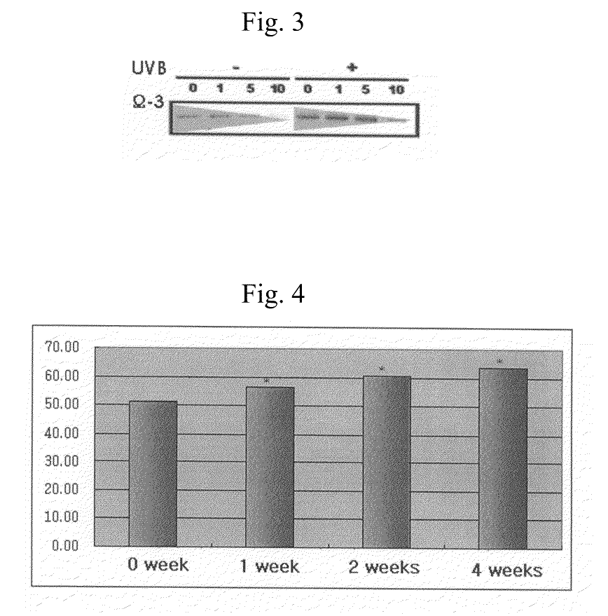

As shown in FIG. 3, it can be seen that the ω-3 fatty acid suppresses the expression of MMP-1 (which is an enzyme decomposing the collagen) derived by UV in a concentration dependent manner. Thereby, it can be seen that the ω-3 fatty acid suppresses the decomposition of the collagen, thereby preventing the skin wrinkle and skin aging. Further, the ω-3 fatty acid promotes the collagen production to improve the wrinkle and to prevent the aging.

### Experimental Example 2

### Clinical Test

In this test, persons to be tested, who were suitable for the test purpose, were selected through the clinical history survey and the skin state diagnosis, and were made to normally use the test product for 4 weeks according to the usage of the product. Before the use, at 1, 2 and 4 weeks after the use, the skin moisture, the keratin improvement, skin roughness and the compact degree of the dermis were measured four times. To this end, following equipments were used (table 3).

TABLE 3

| No. | Measure items | Measure devices | Measure parts |
|---|---|---|---|
| 1 | Face photographing | Facial stage DM-3 | Whole face |
| | | (MORITEX, Japan) | |
| 2 | Skin moisture state | Coroneometer CM825 (C + K, | Right cheek |
| | | Germany) | |
| 3 | Skin keratin state | D-SQUAME tape (Cuderm, | Right and left |
| | | USA), Black tape | cheek |
| | | (MORITEX, Japan) | |
| 4 | Skin roughness state | D-SQUAME tape (Cuderm, | Left cheek |
| | | USA) | |
| 5 | Compact degree of dermis | DermaScan C (Cortex, | Right cheek |
| | | Denmark) | |

In addition, at 1, 2 and 4 weeks after the product use, the persons answered the questions about the efficacy and safety of the product.

In order to examine whether or not the change is significant in statistics before and after the product use, a paired t-test method of SPSS Package Program was used with respect to the measured values (error tolerance<0.05).

The 21 persons took part in the test. One person was dropped out of the test due to the personal reason, and total 20 persons sincerely performed the entire process of the test. Analysis results of the characteristics and skin properties of the persons are as follows (tables 2, 3 and 4). The average age of the persons was 39.1±5. Regarding the skin types, the dry and normal to dry skin types were 25.00% (5 persons), respectively, the normal type was 20.00% (4 persons), the normal to oily type was 15.00% (3 persons) and the oily type was 15.00% (3 persons).

Regarding the moisture state, 50.00% (10 persons) was dry and 45.00% (9 persons) was normal. Regarding the oily state, 20.00% (4 persons) was glossy, 50.00% (10 persons) was normal and 30.00% (6 persons) was deficient. Regarding the skin surface state, 15.00% (3 persons) was soft and 70.00% (14 persons) was normal. Regarding the skin thickness, 70.00% (14 persons) was normal. Regarding the exposure time to the ultraviolet, 35.00% (7 persons) was less than 1 hour, 55.00% (11 persons) was 1-3 hours and 10.00% (2 persons) was 3 hours or more.

Regarding the pigmentation of the skin, the hardly production was 10.00% (2 persons) and the normal was 55.00% (11 persons). The pigmentation was made well in the remaining 35.00% (7 persons).

Regarding the item “the skin is lively?”, 25.00% (5 persons) answered “yes” and 75.00% (15 persons) answered “no.” Regarding the items “the skin is light?” and “the skin elasticity is good?”, only 30.00% (6 persons) answered “yes.” Regarding the item “do you feel skin aging?”, 80.00% (16 persons) answered “yes.”

Regarding the item “stimulus sensitivity”, 75.00% (15 persons) answered “no.” Regarding the item “tingling, sensitivity”, 80.00% (16 persons) answered “no.” Regarding the item “there is a side effect?”, 95.00% (19 persons) answered “no.”

TABLE 4

| No. | name | ages | sexes | dropped? |
|---|---|---|---|---|
| 1 | JSJ | 37 | F | — |
| 2 | HJH | 38 | F | — |
| 3 | HHE | 38 | F | — |
| 4 | CYK | 36 | F | — |
| 5 | SEY | 34 | F | — |
| 6 | JNS | 37 | F | — |
| 7 | KYS | 37 | F | — |
| 8 | CHK | 44 | F | — |
| 9 | SJH | 39 | F | — |
| 10 | KSA | 40 | F | — |
| 11 | KHJ | 29 | F | — |
| 12 | CJA | 45 | F | — |
| 13 | HKY | 46 | F | — |
| 14 | JJO | 44 | F | — |
| 15 | KKS | 46 | F | — |
| 16 | KMJ | 44 | F | — |
| 17 | SYW | 34 | F | — |
| 18 | HEY | 38 | F | — |
| 19 | LHK | 39 | F | — |
| 20 | KJM | 37 | F | — |
| 21 | KES | 40 | F | Yes |
| | | | | interview was |
| | | | | impossible |

TABLE 5

| Items | classification | frequency | percentage (%) |
|---|---|---|---|
| Ages | Twenty something | 1 | 5.00 |
| | Thirty something | 12 | 60.00 |
| | Forty something | 7 | 35.00 |
| skin types | dry | 5 | 25.00 |
| | normal to dry | 5 | 25.00 |
| | normal | 4 | 20.00 |
| | normal to oily | 3 | 15.00 |
| | oily | 3 | 15.00 |
| | problematic skin | 0 | 0.00 |

TABLE 6

| Items | classification | frequency | percentage (%) |
|---|---|---|---|
| Moisture | moist | 1 | 5.00 |
| | normal | 9 | 45.00 |
| | dry | 10 | 50.00 |
| oil | glossy | 4 | 20.00 |
| | normal | 10 | 50.00 |
| | deficient | 6 | 30.00 |
| surface state | soft | 3 | 15.00 |
| | normal | 14 | 70.00 |
| | rough | 3 | 15.00 |
| skin thickness | thin | 4 | 20.00 |
| | normal | 14 | 70.00 |
| | thick | 2 | 10.00 |
| exposure time to | less than 1 hour | 7 | 35.00 |
| UV | 1~3 hours | 11 | 55.00 |
| | 3 hours or more | 2 | 10.00 |
| degree of | hardly | 2 | 10.00 |
| pigmentation | normal | 11 | 55.00 |
| | well | 7 | 35.00 |
| lively? | yes | 5 | 25.00 |
| | no | 15 | 75.00 |
| skin is light? | yes | 6 | 30.00 |
| | no | 14 | 70.00 |
| elastic? | yes | 6 | 30.00 |
| | no | 14 | 70.00 |
| aging? | yes | 16 | 80.00 |
| | no | 4 | 20.00 |
| stimulus sensitivity? | yes | 5 | 25.00 |
| | no | 15 | 75.00 |
| tingling sensitivity? | yes | 4 | 20.00 |
| | no | 16 | 80.00 |
| side effect? | yes | 1 | 5.00 |
| | no | 19 | 95.00 |

(1) Measure of Skin Moisture

The water content of the skin was measured with Corneometer CM 825 (C+K, Germany). As a result of the measure, at 1, 2 and 4 weeks after the product use, the water content of the skin was significantly increased, as compared at the time when the product was not used (table 7, FIG. 4).

TABLE 7

| measured time | Mean | S.D. | SE. Mean | P-value |
|---|---|---|---|---|
| 0 week | 51.33 | 1.9170 | 8.5732 | — |
| 1 week | 56.28 | 1.8673 | 8.3507 | 0.0002* |
| 2 weeks | 60.68 | 1.7228 | 7.7045 | 0.0000* |
| 4 weeks | 63.58 | 1.7341 | 7.7550 | 0.0000* |

(2) Measure of Keratin Amount

In order to analyze the release extent of the keratin and the roughness of the skin surface, the keratin was sampled with Black tape (MORITEX, Japan). The measure part was divided into right and left portions. Then, in the left (right) portion, the keratin layer was sampled with the Black tape, and in the right (left) portion, the keratin was sampled with D-squame tape having Cyanoacrylate applied thereto.

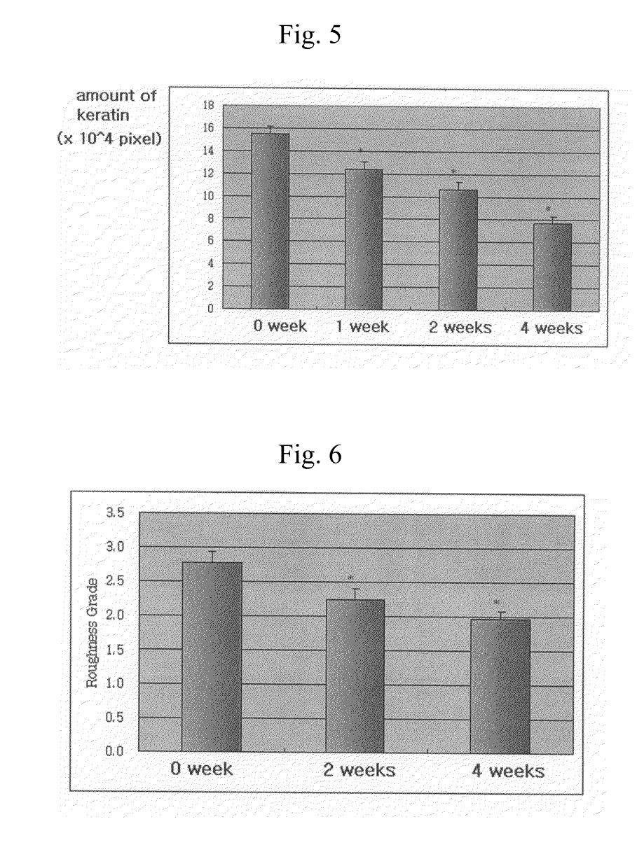

The keratin attached to the Black tape was photographed in a 700 times enlarged digital photograph with Charmview scope (MORITEX, Japan), which was then converted into a bitmap image. With the image, the area of the keratin (pixel number) was obtained with BMI image analysis software (WINaTech, USA). The analyzed values before the sample use and at 2 and 4 weeks after the sample use were compared and analyzed with the paired t-test. As a result of the analysis, the amount of the keratin was significantly decreased from 1 week after the product use until 4 weeks after the product use, as compared to the case where the product was not used (table 8, FIG. 5).

TABLE 8

| measured time | Mean | S.D. | SE. Mean | P-value |
|---|---|---|---|---|
| 0 week | 15.4749 | 3.4806 | 0.7783 | — |
| 1 week | 12.4222 | 3.4042 | 0.7612 | 0.0015* |
| 2 weeks | 10.6448 | 3.5272 | 0.7887 | 0.0002* |
| 4 weeks | 7.6750 | 2.6527 | 0.5932 | 0.0000* |

(3) Measure of the Skin Roughness

The keratin layer of the skin was sampled by dropping Cyanoacrylate to D-squame tape and attaching it on a part to be measured for 1 minute. The sampled keratin layer was coated with Pt/Pd in an ion sputter, which was then observed with a SEM (HITACHI S-4300) by 50 and 100 times. Then, the images were stored. The photographed images of the keratin layer were provided with grades by two skilled tester based on a standard grade, depending on the keratin degrees and the arrangement states of the keratin. The analyzed values before the sample use and at 2 and 4 weeks after the sample use were compared and analyzed with the paired t-test. Then, the decision grades before the sample application were corrected to obtain an improvement rate of the skin. As a result of the analysis, the roughness was significantly decreased in statistics at 2 and 4 weeks after the product use (tables 9 and 10, FIG. 6).

TABLE 9

| decision grades | decision standards |
|---|---|
| 1 | There occurs no keratin |
| 2 | There occurs a little small keratin |
| 3 | The small keratin occurs on the whole |
| 4 | Conglomerated keratin occurs |
| 5 | The large conglomerated keratin occurs on the whole |

TABLE 10

| Measured time | Mean | S.D. | SE. Mean | P-value |
|---|---|---|---|---|
| 0 week | 2.7750 | 0.7518 | 0.1681 | — |
| 2 weeks | 2.2500 | 0.6977 | 0.1560 | 0.0153* |
| 4 weeks | 1.9750 | 0.4993 | 0.1117 | 0.0006* |

(4) Compact Degrees of the Dermis

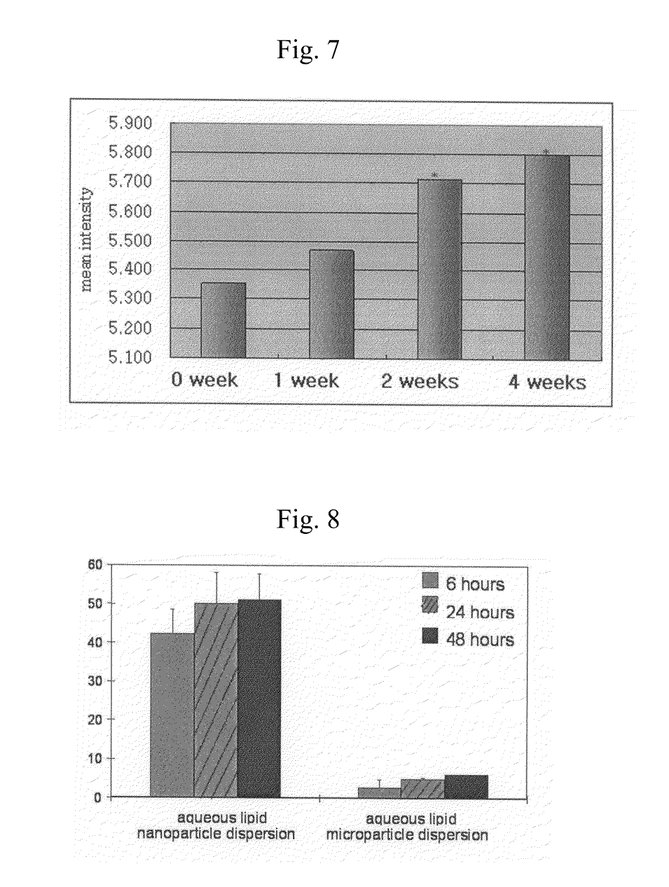

The ultrasonic photographing was performed with DermaScanC® available from a Cortex company, thereby measuring the compact degrees of the dermis. As a result of the measure, the compact degrees of the dermis were significantly increased at 2 and 4 weeks after the product use, as compared to the case where the product was not used (table 11, FIG. 7).

TABLE 11

| measured time | Mean | S.D. | SE. Mean | P-value |
|---|---|---|---|---|
| 0 week | 5.353 | 0.1245 | 0.5568 | — |
| 1 week | 5.468 | 0.1773 | 0.7930 | 0.2392 |
| 2 weeks | 5.715 | 0.1998 | 0.8935 | 0.0169* |
| 4 weeks | 5.800 | 0.2056 | 0.9195 | 0.0071* |

(4) Survey Assessment

At 1, 2 and 4 weeks after the product use, the survey of the product usability and satisfaction was carried out. Based on the survey, the affirmative answers are arranged for each assessment time point, as follows.

TABLE 12

| Items | 1 week | 2 weeks | 4 weeks |
|---|---|---|---|
| it became moist | 3.45 | 3.65 (▴5.80%) | 4.10 (▴18.84%) |
| Retraction was reduced | 3.40 | 3.60 (▴5.88%) | 4.25 (▴25.00%) |
| Became glossy | 3.35 | 3.45 (▴2.99%) | 3.75 (▴11.94%) |
| Became softened | 3.50 | 3.65 (▴4.29%) | 3.95 (▴12.86%) |
| keratin was decreased | 3.30 | 3.40 (▴3.03%) | 3.65 (▴10.61%) |
| skin became delicate | 3.50 | 3.80 (▴8.57%) | 4.00 (▴14.29%) |
| accordion pleats | 2.95 | 3.10 (▴5.08%) | 3.35 (▴13.56%) |
| (wrinkles) were | | | |
| reduced | | | |
| elasticity was increased | 3.10 | 3.25 (▴4.84%) | 3.60 (▴16.13%) |
| skin color became lighter | 3.05 | 3.45 (▴13.11%) | 3.80 (▴24.59%) |
| paste of makeup is good | 3.40 | 3.80 (▴11.76%) | 4.30 (▴26.47%) |
| skin became healthy | 3.15 | 3.45 (▴9.52%) | 3.75 (▴19.05%) |
| skin is lively | 3.10 | 3.40 (▴9.68%) | 3.75 (▴20.97%) |

▴(after the use − before the use)/before the use * 100

The scores in Table 13 below represent frequencies of subjects who expressed satisfaction among the 20 subjects.

TABLE 13

| Items | Frequency | % |
|---|---|---|
| Color | 18 | 90 |
| Scent | 13 | 65 |
| Applicability | 19 | 95 |
| Absorption | 18 | 90 |
| Satisfaction | 18 | 90 |

### Experimental Example 3

### Comparison Test of Transport Systems

(1) Skin Permeation

An aqueous lipid nano-particle dispersion (1) and an aqueous lipid micro-particle dispersion (2) were prepared according to Table 14. The compositions of two dispersions are the same as each other except that the contents of the Tego Care 450 are different from each other. The aqueous lipid nano-particle dispersion (1) was prepared by splitting the particles to nano-sizes using a high-pressure emulsifier. The aqueous lipid micro-particle dispersion (2) was prepared by splitting the particles to micro-sizes using an emulsifier.

TABLE 14

| Components | 1 | 2 |
|---|---|---|
| Black currant oil | 20.0 | 20.0 |
| Carnauba wax | 20.0 | 20.0 |
| Tego Care 450 | 5.0 | 1.0 |
| Purified water | To 100 | To 100 |
| Glycerin | 20.0 | 20.0 |
| Phenonip (preservative) | 0.1 | 0.1 |

The skin permeability was measured as follows:

The skin of a hairless mouse (6-8 weeks age) was cut to be 2×2 cm² and was fixed in Franz-type diffusion cell (inner diameter: 9 mm) after covering and capping the skin. 0.01M phosphate buffer (pH 7.4) was poured to the receptor part and the temperature of the diffusion cell was maintained to be 37±0.1° C. 50 μl of test materials were applied to stratum corneum layers and then the receptor solution (phosphate buffer solution) was stirred at 600 rpm. After 6, 24, 48 hours, 0.5 ml solution was recovered from the receptor part, 0.5 ml of a new phosphate buffer solution was supplemented. The recovered sample was subject to LSC (liquid solid chromatography) to measure concentrations. After 24 hours, the skin was detached from the diffusion cell and cleaned with cotton four times. Then, the skin was subject to Transpore® (3M Tape) treatment. The 1^{st} strip was discarded and subsequent 10 tape strippings were carried out serially, and put to scintillation vials. The remaining skin samples were also put to scintillation vials after cutting the application parts of the test materials. 2.5 ml of scintillation cocktail was added to each of the vials and sufficient votex was carried out. Then, LSC measurement was carried out. The result was shown in FIG. 8.

As shown in FIG. 8, the skin permeation of the transport system according to an embodiment of the invention was best.

(2) Effect Continuation

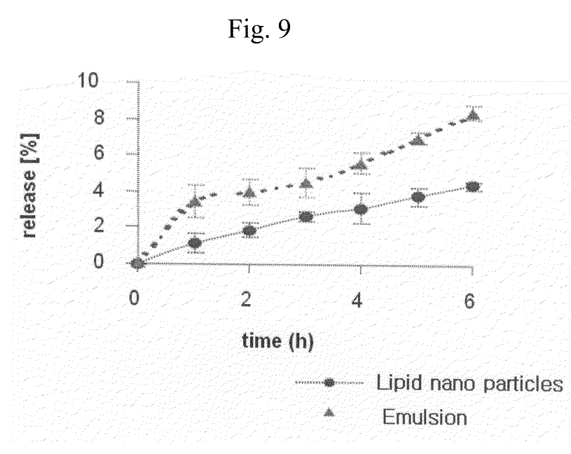

Formulations were prepared according to Table 15 below. “1” was prepared in a form of nano-particles of black currant oil by using solid carnauba wax lipid. “2” was prepared by emulsifying using mineral oil. Then, the release amounts of black currant oil, which is an effective ingredient, were measured for the “1” and “2.” The result was shown in FIG. 9. As shown in FIG. 9, the drug release became slow because of generation of particles by the ligid solid lipid, and therefore, the effect can be maintained longer.

TABLE 15

| Components | 1 | 2 |
|---|---|---|
| Black currant oil | 20.0 | 20.0 |
| Carnauba wax | 20.0 | — |
| Mineral oil | — | 20.0 |
| Tego Care 450 | 5.0 | 5.0 |
| Purified water | To 100 | To 100 |
| Glycerin | 20.0 | 20.0 |
| phenonip (preservative) | 0.1 | 0.1 |

(3) Water Content

According to the compositions of Table 16 below, “1” including 10% lipid nano-particle dispersion containing ω-3 fatty acid was prepared. The dispersion (1) is the same as that of “1” of Table 14.40% of the dispersion is oil phase and accordingly oil part based on the total formulation weight occupies 4%. “2” was prepared by adding 4% of isopropanol and “3” was prepared by adding 4% paraffin (the amounts of total oil phase are the same as that of “1”).

The oil phase was dissolved at 70° C. in a separate vessel and dispersed using a homogenizer to form a hydrophobic mixture. The aqueous phase was also dissolved at 70□, the hydrophobic mixture was slowly added to the aqueous phase and emulsified at 70° C. The mixture was cooled in an ice bath after the emulsification and a preservative was added. Finally, the emulsion composition was poured to a sealed vessel and cooled to a room temperature using a cooler.

TABLE 16

| | Components | 1 | 2 | 3 |
|---|---|---|---|---|
| Oil phase | Stearic acid | 1.0 | 1.0 | 1.0 |
| | Cetearyl alcohol | 0.2 | 0.2 | 0.2 |
| | Hydrogenated oil | 0.7 | 0.7 | 0.7 |
| | Hydrophobic glyceryl stearate | 0.5 | 0.5 | 0.5 |
| | Hydrogenated lecithin/C12-16 | 1.0 | 1.0 | 1.0 |
| | alcohol/palmitic acid | | | |
| | Preservatives | 0.3 | 0.3 | 0.3 |
| | Polydecen | 4.0 | 4.0 | 4.0 |
| | Meadow foam seed oil | 1.0 | 1.0 | 1.0 |
| | Pentaerythrityl tetraisostearate | 3.0 | 3.0 | 3.0 |
| | Dimethicone | 0.5 | 0.5 | 0.5 |
| | Cetyl octanoate | 2.0 | 2.0 | 2.0 |
| | Isopropanol | — | 4.0 | — |
| | Paraffin | — | — | 4.0 |
| | Cyclodimethicone | 3.0 | 3.0 | 3.0 |
| Aqueous | Purified water | To 100 | To 100 | To 100 |
| phase 1 | Glycerin | 4.0 | 4.0 | 4.0 |
| | Butylenes glycol | 5.0 | 5.0 | 5.0 |
| | Disodium EDTA | 0.02 | 0.02 | 0.02 |
| | TEA(triethanolamine) | 0.15 | 0.15 | 0.15 |
| | Preservatives | 0.2 | 0.2 | 0.2 |
| | Lipid nano-particle dispersion | 10.0 | — | — |
| | containing ω-3 fatty acid | | | |
| Aqueous | Carbomer 1% aqueous solution | 12.0 | 12.0 | 12.0 |
| phase 2 | | | | |

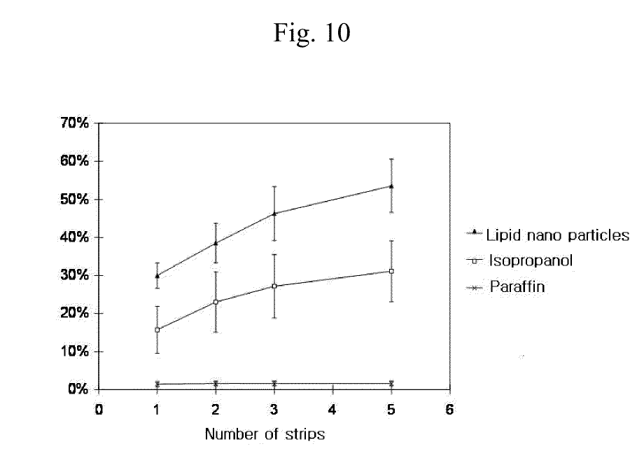

As shown in FIG. 10, the water content of the transport system according to an embodiment of the invention was highest.

### Experimental Example 4

### Measurement of Generation Amount of Ceramide

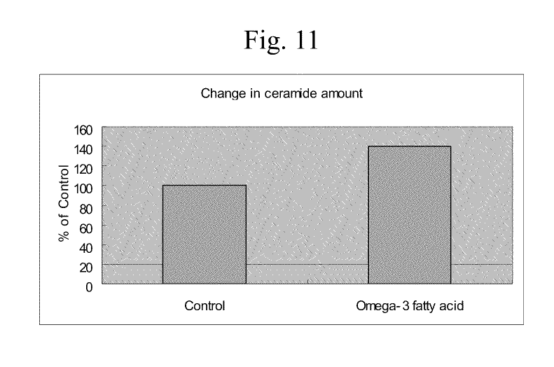

Normal human keratinocytes obtained from a new-born baby (passage 3, manufactured by the inventors) were cultured in a KBM medium (0.03 mM Ca²⁺, Clonetics, US) in a 75 T culture flask. A KBM medium supplemented with insulin, hydrocortisone, BPE (bovine pituitary extract), hEGF, etc. was used. After culturing for 3 days, the cells were treated with test materials in a medium wherein BPF was removed. While culturing them at 37° C. (5% CO₂), the cells were harvested at the 3^{rd} day and the 6^{th} day and stored in a refrigerator of −20° C. Lipid was extracted, and subject to TLC and densitometer (CAMAG company) to rate. The result was shown FIG. 11. As shown in FIG. 11, when the keratinocytes were treated with omega-3 fatty acid, even more ceramide was produced than the control. Ceramide may restore and strengthen skin barrier function. Therefore, omega-3 fatty acid was proved to have an effect of restoring and strengthening skin barrier function.

### Experimental Example 5

### Measurement of Change of Elastin

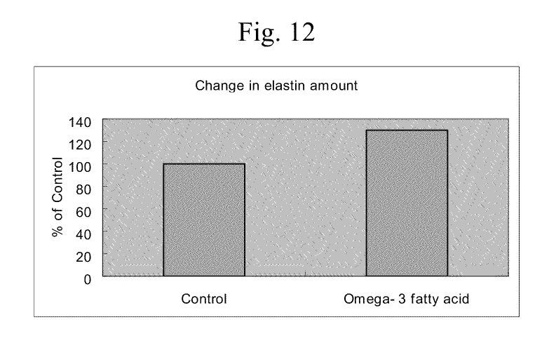

In order to measure elasticity change by the composition according to the present invention, Rhino mouse (female, 10 weeks age), of which skin drooped due to decrease of elasticity, was treated with a control and omega-3 fatty acid on its back for 14 days. The elasticity change was observed with eyes and the change in the amount of elastin in the dermal layer was also measured. The result was shown in FIG. 12. As shown in FIG. 12, when omega-3 fatty acid was applied, the amount of elasin increased.

### Experimental Example 6

### Restoration and Prevention of Skin Damage Caused by UV

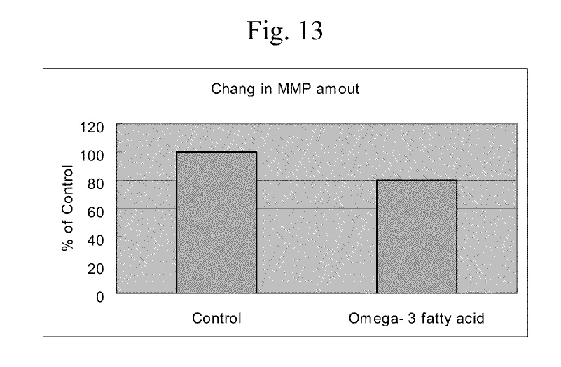

Test materials were applied to the back of a hairless mouse while radiating UV. If UV is radiated to skin, MMP (matrix metalo protease), which is a collagenase, increases and accordingly collagen decreases and wrinkles are created. The back of the hairless mouse was treated with the control and the omega-3 fatty acid for 10 weeks. The test materials were applied immediately after the UV radiation. Biopsy of the tissues were made after 12 weeks and the changes in MMP amounts in skin were measured. The result was shown in FIG. 13. As shown in FIG. 13, MMP was decreased by omega-3 fatty acid.

As described above, according to the invention, it is possible to restore and, strengthen the damaged and disordered skin barrier and to put life into the tired and depleted cells. In addition, it is possible to protect the skin against the exterior harmful environments and to prevent the skin water from being emitted to the outside. Furthermore, it is possible to defend the skin against the harmful factors in the skin, thereby regenerating the collagen in the damaged and deficient skin. Moreover, it is possible to increase the absorption of the effective ingredients into the skin and to enable the effective ingredients to be correctly transported to the target part. In addition, a stable preparation containing omega-3 fatty acid as an effective ingredient can be provided.

While the invention has been shown and described with reference to certain preferred embodiments thereof, it will be understood by those skilled in the art that various changes in form and details may be made thereto without departing from the spirit and scope of the invention as defined by the appended claims.

What is claimed is:

1. A method for protecting skin and improving skin state comprising administering an effective amount of a composition for skin external use containing omega-3 fatty acid as an effective ingredient to a subject in such need.

2. The method according to claim 1, which protects skin and improves skin state by preventing or alleviating skin aging.

3. The method according to claim 1, which protects skin and improves skin state by moisturizing skin.

4. The method according to claim 1, which protects skin and improves skin state by improving skin roughness.

5. The method according to claim 1, which protects skin and improves skin state by restoring or strengthening skin barrier function

6. The method according to claim 1, which protects skin and improves skin state by improving skin elasticity.

7. The method according to claim 1, which protects skin and improves skin state by recovering or preventing a skin damage due to UV.

8. The method according to claim 1, wherein the omega-3 fatty acid is administered in combination of an effective amount(s) of Rosevita extract and/or copper peptide.

9. The method according to claim 1, wherein the omega-3 fatty acid is administered in the form of black currant oil.

10. The method according to claim 1, wherein the omega-3 fatty acid is administered transdermally.
